(19) United States
(12) Patent Application Publication
(10) Pub. No.: US 2004/0033503 A1
(43) Pub. Date: Feb. 19, 2004

(54) Method for attaching oligonucleotide to solid support and the oligonucleotide array prepared by the method thereof

(76) Inventors: Jae-Jong Kim, Taejon (KR); Sun-Ho Cha, Taejon (KR)

Correspondence Address: CHERYL H AGRIS PHD, PO BOX 806, PELHAM, NY, 10803, US

(21) Appl. No.: 10/257,528

(22) PCT Filed: Mar. 29, 2001
(86) PCT No.: PCT/KR01/00514

(51) Int. Cl. C12Q 1/68; C12M 1/34; B05D 3/00

(52) U.S. Cl. 435/6; 435/287.2; 427/2.11

(57) ABSTRACT

The present invention relates to a method for attaching oligonucleotide to a solid support and an oligonucleotide array prepared by said method. In the present invention, an oligonucleotide array is prepared by the steps of: preparing a hydrophobic attachment layer; spotting solution of oligonucleotide in which a hydrophobic group is bonded to one side of its terminal onto the hydrophobic attachment layer; and solidifying the hydrophobic attachment layer.

### TECHNICAL FIELD

The present invention relates to a method of attaching oligonucleotide to a support, and to an oligonucleotide array prepared by means of said method. The present invention can be effectively used in the areas of genetic diagnosis and analysis, and DNA chips, which are based on hybridization.

### BACKGROUND ART

Hybridization, which uses oligonucleotide attached to a support, is being widely used in all related areas of biotechnology as a method of searching for various types of genes. Recently, based on this basic principle, a high-density direct oligonucleotide array, or a DNA chip has started to replace the traditional gel-based methods of the prior art. It is being put into practical use while rapidly becoming a tool for fast and economical determination of genetic mutation, expression of genetic characters, DNA sequence, etc. However, there is a difficulty as to the method of effectively attaching oligonucleotides to the support.

Up to now, various methods have been disclosed, which involve attaching pre-synthesized oligonucleotide onto a support such as glass, silicon, a nitrocellulose film, or polystyrene, or synthesizing oligonucleotide directly onto the support.

With respect to the method of synthesizing oligonucleotide directly onto a support, it involves arraying oligonucleotide of 25 mer or below at high density. Nevertheless, in many areas such as diagnosis, a gene fragment prepared by pre-synthesized oligonucleotide, PCR, etc. must be used. For this task, two methods have been proposed. First, there is the method of using mutual covalent bond by respectively activating the DNA and the surface of the support with a bond reacting group. Second, there is the bonding method, such as passive adsorption, avidin-biotin affinity bonding, or the method of mixing oligonucleotide with polyacrylamide, polypyrol, or nitrocellulose solution, followed by co-polymerization. Further, there is the method of a three-dimensional functional polyacrylamide gel pad, which involves inducing covalent bond with oligonucleotide by forming a polyacrylamide gel onto a glass plate, followed by activation of the gel surface.

More specifically, in Duggan et al., a DNA fragment was bonded onto a glass plate by way of covalent bond between the functional groups of the surface of the glass plate and the thymidine residues of DNA. This was accomplished by inducing functional groups having positive electric charge by coating the surface of the glass plate with polylysine and then spotting the DNA solution, followed by irradiation of UV (Duggan, D. J. et al. (1999) Nat Genet. (Suppl.) 21, 10-14). However, the DNA attached to the support according to this method is attached non-specifically to the support without specific directions, and therefore affects the hybridization therein.

To minimize the occurrence of nonspecific bond as such, Chrisey et al. carried out DNA attachment and hybridization by using oligonucleotide modified by adding thiol to one direction, a silica slide, or an n-type Si wafer support modified by adding aminosilane, and various types of amine/sulfhydril heterobifunctional crosslinkers. As reported, the method resulted in attachment density of 162-234 fmol/mm², and hybridization efficiency of 9.3-76.1% (15-178 fmol/mm²), Linda A. Chrisey et al. (1996) Nucleic Acids Res. 24, 3031-3039. However, in case of an oligonucleotide comprising various types of bases, the method results in low efficiency, which in turn limits the utility thereof depending on the types of oligonucleotides. Moreover, Roger et al. reported the method of direct attachment by using a disulfidized-modified oligonucleotide with a d(T)₁₀ spacer without crosslinkers onto a support modified by mercaptosilane (Yu-Hui Roger et al. (1999) Anal. BioChem., 266 23-30). The method resulted in high, attachment density (3×10⁵ oligo/um²=500 fmol/mm²) and high hybridization efficiency (16%). Furthermore, it reported that hydrophobic characteristics of the support surface could work to suppress the “doughnut” phenomenon, which is formed therein depending on the dry condition after the spotting, and also the mixture phenomenon, which can occur while simultaneously spotting various types of oligonucleotides at a high density.

Joos et al. disclosed an attachment method using an amino/carboxy heterobifunctional crosslinker (EDC) between the carboxylized oligonucleotide and the support modified by the amino group (Beda Joos et al. (1997) Anal. Biochem., 247, 96101). According to Joos, the attachment efficiency at attachment was different according to the pH of the EDC reaction, and its maximum attachment density was 160 fmole/mm². The optimum condition for hybridization required approximately 15 or more of bases as a spacer with respect to the attached oligonucleotide.

As another example requiring a spacer, Guo et al achieved covalent bond with amino-modified oligonucleotide by treating the support with aminoprophyltrimethoxylsilane and then reacting the same with pphenylenediisothiocynate (Zhen Guo et al. (1994) Nucleic Acids Res. 22, 5456-5465). According to Guo et al., the maximum attachment density was 330 fmole/mm² and used d(T)₁₅ as a spacer.

These types of methods utilizing covalent bond, in most parts, require preparation of a separate crosslinker and pre-treatment to induce reacting groups which can mutually bond respectively to the surface of a solid medium (equivalent to a “support” according to the present invention, and the term is used interchangeably therewith). Hence, the production process thereof is complex with an increase in production cost for oligonucleotide arrays.

As for the method of passive adsorption, which is a non-covalent bond method, the method of using polystyrene as a support has been presented (Nikiforov T. T. et al. (1995) Anal Biochem., 227, 201-209). It is a method of attachment, which involves reacting the DNA solution containing base or a cationic surfactant. In this manner, the base is able to reduce the repulsive force between the negative charge of phosphate of the oligonucleotide backbone and the polystyrene surface. By reducing the repulsive force between the negative charges of the oligonucleotides, the positive charge of a cationic surfactant can minimize the electric repulsive force between polystyrene and oligonucleotide, thereby inducing bondings by hydrophobic interactions. The attachment efficiency of oligonucleotide, achieved by this method was 1.1 pmol/well, and hybridization went up to 50% at its maximum. This method is comparatively simple and economical. Yet, there is a limitation with respect to the availability of supports for commercial use, and also the method brings about unstable attachment at a high temperature.

Further, as for the method of using biotin-avidin affinity bonding (Syvanen, A. C. et al. (1998) Nucleic Acids Res. 16, 11327-11338), it involves a pre-treatment step of bonding avidine and biotin, respectively, to the solid medium and to the oligonucleotide. As such, the unit production cost of the sample becomes very high.

The co-polymerization method involves mixing oligonucleotide with an acrylamide group at its 5-terminal in acrylamide and spotting this solution onto a glass slide treated with silane, followed by co-polymerization. In this manner, the polymer layer with a base portion of oligonucleotide exposed to the outside was formed onto the surface of glass (Farah N. Rehman et al. (1999) Nucleic Acids Res. 27, 649-655). This method results in attachment efficiency of 83-84% (approximately 200 fmol/mm²), which is very high. However, hybridization was approximately 15% under the aforementioned conditions of attachment, which is relatively low.

### DISCLOSURE OF THE INVENTION

Accordingly, overcoming the disadvantages of the prior art, the present invention seeks to provide a method of attaching oligonucleotide to a support for bonding in the specific direction for the purpose of hybridization, with high bonding efficiency and stability. The present invention also provides an oligonucleotide array prepared by said method.

Moreover, with respect to a large variety of oligonucleotides, the present invention seeks to provide a method of attaching oligonucleotide, which can be used without limitation, in addition to an oligonucleotide array prepared by said method.

Further, the present invention seeks to provide a method of attaching oligonucleotide to a support, which is simple and economical, and an oligonucleotide array prepared by said method.

### BRIEF DESCRIPTION OF DRAWINGS

FIG. 1 is a photograph showing the results of hybridization of DMT oligonucleotide attached to a support and those of generic oligonucleotides.

FIG. 2 is a photograph showing the results of hybridization of DMT oligonucleotide attached to a support by concentration by means of complementary and non-complementary fluorescent oligonucleotides.

FIG. 3 is a photograph confirming reproducibility by carrying out hybridization after attaching DMT oligonucleotide onto a support under the same conditions.

FIG. 4 shows the standard curve of fluorescent size for confirming the attachment rate and hybridization efficiency.

FIG. 5 is a photograph showing the results of hybridization of DMT oligonucleotide attached to a support by concentration by means of with complementary fluorescent oligonucleotides.

FIG. 6 conceptually illustrates the method of attaching oligonucleotide to a support.

FIG. 7 is a photograph showing the results of hybridization with complementary and non-complementary fluorescent oligonucleotides after attaching pyrene oligonucleotide and generic oligonucleotide to a support.

FIG. 8 is a photograph showing the results of hybridization with fluorescent oligonucleotides after attaching four types of cholesterol oligonucleotide and generic oligonucleotide to a support.

FIG. 9 is a photograph showing the results of hybridization with complementary and non-complementary fluorescent oligonucleotides after attaching three types of cholesterol oligonucleotide and generic oligonucleotide to a support.

### BEST MODE FOR CARRYING OUT THE INVENTION

The present invention provides an improved method of attaching oligonucleotide to a support. Moreover, it provides an oligonucleotide array prepared by said method.

In achieving said objectives, the present invention comprises the following steps of: applying a hydrophobic attachment layer onto a support, which can be solidified under certain conditions; spotting certain portions of said attachment layer with aqueous oligonucleotide solution in which hydrophobic groups have bonded to 3′- or 5′ terminals; and solidifying said attachment layer.

Moreover, the present invention comprises the following steps of: fluidifying the solidified hydrophobic attachment layer applied onto said support under certain conditions; spotting certain portions of said attachment layer with oligonucleotide aqueous solution in which hydrophobic groups have bonded to 3′- or 5′ terminals; and solidifying said attachment layer.

Further, the present invention includes a method of attaching PCR products amplified from the primers in which hydrophobic groups have bonded to 5′-terminals, instead of the oligonucleotide in which hydrophobic groups have bonded to said 3-terminals or 5′-terminals.

With respect to the present invention, it is characterized by polymers preferably formed by polymerization or condensation polymerization onto said attachment layer.

With respect to the present invention, the hydrophobic group bonding to oligonucleotide, a PCR amplified product, or the primer thereof is preferably a dimethoxytrityl group, pyrene, or cholesterol.

In addition, the present invention comprises an oligonucleotide array prepared by said method, or more particularly, an oligonucleotide array in which hydrophobic groups have been attached to 5′-terminals or 3′-termials of a multiple of oligonucleotides at the attachment layer applied onto the support.

As for the support of the present invention, any type of supports used in DNA chips can be used, e.g., glass, silicon, nitrocellulose films, or polymers such as polystyrene.

The term “oligonucleotide” in the present invention means a molecule of two or more, up to several tens of various types, of nucleic acids in bond, including adenine, guanine, cytosine, thiamine, uracil, and the derivatives thereof. In addition, the term also includes PCR products amplified by primers.

The DNA synthesis is accomplished by repetition of a series of the following four steps of: (1) deprotection for activating 5′-OH by removing the protection group of 5′-OH of oligonucleotide of a base monomer, dimer or more (e.g., dimethoxytrityl group (DMT)); (2) coupling for forming a phosphitetriester bond between the deprotected 5′-OH and the new base monomer; (3) oxidation for changing the phosphitetriester bond into a stable phosphorus; and (4) capping for inactivation by using a dimethoxytriltyl group (DMT) onto the remaining unreacted 5′-OH (the concept is equivalent to “protection” herein). Consequently, the 5′-OH of an oligonucleotide thus finally synthesized is protected is by a protection group. In general, in case of an oligonucleotide, it is synthesized after undergoing a “deprotection step,” or it is used after removing the 5′-protection group thereof.

As for the attachment layer of the present invention, it includes a variety of hydrophobic substances, which can be solidified under certain conditions. Preferably, polymers formed by polymerization or condensation polymerization would be used. The attachment layer is formed by dissolving the monomers, each of which is a unit of said polymers, into an appropriate solvent to the state of fluidity and then applying the same onto a support. For example, PVC resins or polymers which can be solidified by UV or light may used.

According to the present invention, after the application onto the support but before its solidification, the attachment layer is spotted with the oligonucleotide solution in which a hydrophobic group has bonded to one side of the terminal, respectively. Then, hydrophobic interaction is allowed to run its course by way of hydrophobic groups within the hydrophobic attachment layer. Thereafter, the attachment layer can be solidified by polymerization, etc. The method of polymerization or solidification of the attachment can be selected according to the characteristics of the attachment layer as used therein. For example, it can be solidified by drying, irradiation of UV etc.

Further, if it is necessary to attach the oligonucleotide after pre-applying the attachment layer onto a support, followed by solidification, the solidified hydrophobic attachment layer which has been applied onto the support can be fludifiied under certain conditions, for example, by means of the method of adding a certain amount of organic solvent, and then the oligonucleotide solution in which a hydrophobic group has been bonded to one side of the terminal, respectively, can be spotted onto the certain portions of said attachment layer, followed by re-solidification of said attachment layer.

As for the solvent for dissolving oligonucleotide in the present invention, any hydrophilic solvents which dissolve oligonucleotide can be used. For example, water or appropriate buffer solution can be used. In particular, it is preferable to use a solvent which can stabilize oligonucleotide, and 3′- or 5′-terminal hydrophobic groups of oligonucleotide.

In the present invention, the oligonucleotide therein has a hydrophobic group at its 3′- or 5′-terminal. In one embodiment of the present invention, after the synthesis of oligonucleotide, it can be used in the preparation of an oligonucleotide array without removing the hydrophobic protection group attached to its 5′-terminal. As examples, the DNA monomer for producing oligonucleotide, currently in commercial use, usually has its 5′-terminal protected by DMT, and in this case, after the production of oligonucleotide, it can be used without removing DMT attached to the 5′-terminal of oligonucleotide bonded in the final step. In such a case, by omitting the process of DMT removal (i.e., the process of deprotection, and the process of bonding to other linkers), the process in its entirety is substantially simpler as compared to those of the prior art, with a significant reduction of costs. If necessary, the protection group of the 5′-terminal remaining after the production of oligonucleotide should be removed, and the other hydrophobic protection group bonded to one side of its 3′- or 5′-termianl, after which it can be used in the present invention.

Moreover, if necessary, the hydrophobic group to be bonded to one side of the terminal of oligonucleotide can be bonded to the 3′- or 5′-terminal. For example, if it is necessary to bond oligonucleotide onto a support in the specific direction, the hydrophobic group can be bonded to its 3′-terminal or 5′-terminal to set the direction of oligonucleotide attached to the support.

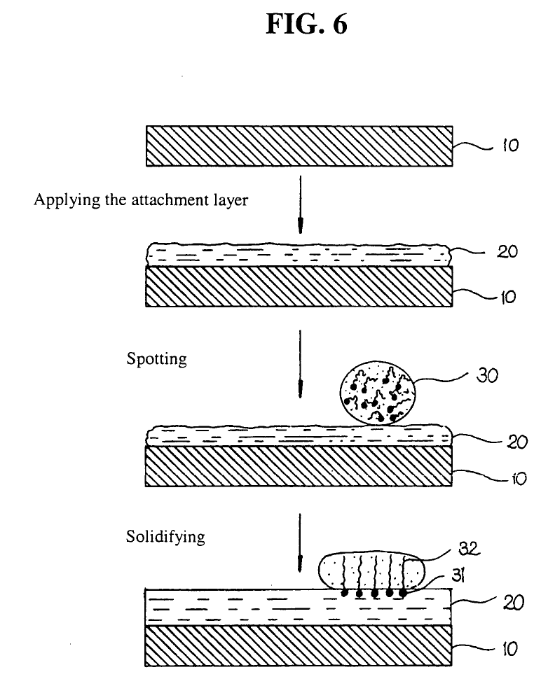

In the present invention, in using an oligonucleotide bonded to a hydrophobic group at one side of its terminal, the attachment layer is supplied with the solution in which these hydrophobic groups have been bonded to the oligonucleotide. There, with respect to its hydrophilicity and hydrophobicity, due to the affinity between those of the same disposition and the repulsive force between those of the different disposition, the region of the hydrophobic group bonded to the oligonucleotide is relatively situated toward the hydrophobic attachment layer, and the hydrophobic region of oligonucleotide (i.e., DNA) is maintained in the direction opposite of the attachment layer (refer to FIG. 6). First, the attachment layer 20 is applied onto the support 10. If the attachment is in the state of fluidity (e.g., when the monomers are not polymerized), the oligonucleotide solution is spotted onto the attachment layer. At that time, the hydrophobic group 31 bonded to one side of the terminal of oligonucleotide is hydrophobically bonded onto the hydrophobic attachment layer, and the hydrophilic oligonucleotide side 32 becomes situated in the direction opposite of the support. The oligonucleotide becomes attached onto the support by way of solidification of the attachment layer through the solidification steps.

By drying (or solidifying) the attachment layer in this state, the oligonucleotide having an optimal direction for hybridization is attached to the attachment layer on the support. Consequently, with respect to the oligonucleotide array prepared according to the present invention, the hydrophobic portions of 5′-terminals and 3′-terminals are accurately attached to the support side. There, since the oligonucleotide is situated on the opposite side of the support, another point of advantage is that there is no adverse impact by way of steric hindrance during its hybridization.

### EXAMPLE 1

### Preparation of Oligonucleotide

The oligonucleotide with the following sequence was synthesized by using a synthesizer of 8089 Expedite Nucleic Acid Synthesis System of PerSeptive Biosystems, Inc.

The sequence as follows is an artificial sequence of an arbitrary selection, but the present invention can be applied to all types of oligonucleotides, irrespective of the sequence characteristics of an oligonucleotide.

(1) Generic Oligonucleotide

It was used as a control for the attachment and hybridization of an oligonucleotide having a hydrophobic group. It was synthesized and purified as follows: After synthesizing the oligonucleotide to the state of trityl-ON, in which a hydrophobic group is protected by DMT, it was treated with the ammonia solution to separate it from the support used during its synthesis. Then, by using a COP column, it was purified to the state in which a DMT group has been removed. It was quantified with a spectrometer, and after completely drying the same with a vacuum condenser, it was dissolved with 1×TE buffer solution at pH 8 to the concentrations of 100 and 200 pmol/ul, respectively, and kept in a freezer for future use.

The oligonucleotide synthesized and purified by said method did not have any types of hydrophobic groups, but had the same base sequence as an oligonucleotide having a hydrophobic group (DMT- and cholesterol oligonucleotides, respectively). Types and base sequences of generic oligonucleotide were as follows:

No. 1:
5′-GCT TTG GGG CAT GGA CAT TGA CCC GTA TAA-3′
(30 mer)
No. 2:
5′-GCT TTG GGG CAT GGT TAT TGA CCC GTA TAA-3′
(30 mer)
No. 3:
5′-TTT TTC TGG GCC TGT GGC TGG-3′
(21 mer)
No. 4:
5′-TTT TTT ATG GGG ATA TGC TGG TGA G-3′
(25 Omer)
No. 5:
5′-TTT TTT CCG CCT CAC AGT TGA TGG A-3′
(25 mer)

(2) Oligonucleotide with a Hydrophobic Group

(A) DMT Oligonucleotide

Like the generic oligonucleotide, after synthesizing the oligonucleotide, in which its 5′-terminal is protected by DMT, to the state of trityl-ON, it was treated with ammonia solution. Then, an appropriate amount of 1M TRIS buffer solution (pH 8) was added thereto to the final concentration of 100 mM. Then, it was dried with a vacuum condenser and dissolved with 1×TE buffer solution at pH 8. It was quantified with a spectrometer and then diluted to various concentrations of 100 and 200 pmol/ul, respectively, after which were kept in a freezer for future use. Types and base sequences of DMT oligonucleotides were as follows:

DMT 1:
5′-DMT-GCT TTG GGG CAT GGA CAT TGA CCC GTA TAA-3′
(30 mer)
DMT 2:
5′-DMT-GCT TTG GGG CAT GGT TAT TGA CCC GTA TAA-3′
(30 mer)

DMT 1 and 2 of DMT oligonucleotides had the same base compositions as generic oligonucleotide No. 1 and 2, respectively, and they were distinguished according to the existence of a hydrophobic DMT group at the 5′-terminal.

(3) Pyrene-Oligonucleotide

By using pyrene-CE phosphoramidite (Cruachem), the modified pyrene oligonucleotide was trityl-OFF synthesized by means of pyrene-bonding the side of a 5-terminal. Thereafter, it was purified with RP-HPLC and dried with a vacuum condenser. Then, it was made into 500 pmol/ul in 1×TE buffer solution at pH 8, which was kept in a freezer for future use. Since pyrene-oligonucleotide has strong hydrophobicity, it could be purified by RP-HPLC after trityl-OFF synthesis. The base composition of pyrene-oligonucleotide was as follows:

Pyr. 1: 5′-pyrene-CTT CTG ACT TCT TTC CTT CTA TTC-3′ (24 mer)

(C) Cholesterol Oligonucleotide

By using cholesterol phosphoramidte (Chemgenes), the modified cholesterol oligonucleotide was trityl-OFF synthesized by bonding cholesterol to a 5-′terminal, followed by RP-HPLC purification. The purified cholesterol oligonucleotide was dried with a vacuum condenser. Then, it was made into 500 pmol/ul in 1×TE buffer solution at pH 8, which was kept in a freezer for future use. The types and the base compositions of cholesterol oligonucleotides were as follows:

Chol. 1: 5′-chorestrol-CTT CTG ACT TCT TTC CTT CTA TTC-3′ (24 mer)
Chol. 2: 5′-chorestrol-TTT TCC TGG GCC TGT GGC TGG-3′ (21 mer)
Chol. 3: 5′-chorestrol-TTT TTT ATG GGG ATA TGC TGG TGA G-3′ (25 mer)
Chol. 4: 5′-chorestrol-TTT TTT CCG CCT CAC ACT TGA TGG A-3′ (25 mer)

Cholesterol oligonucleotides, Chol. 2, 3 and 4 had the same base sequences as generic oligonucleotides No. 3, 4, and 5, respectively.

(3) Fluorescent Oligonucleotide

It was used in confirming hybridization, and the oligonucleotide having its 5′-terminal modified to fluorescein was synthesized by using fluorescein-CE phosphoramidite (Cruachem), followed by PAGE purification. The oligonucleotide having its 3′-terminal modified to fluorescein was trityl-ON synthesized by using fluorescein column (Cruachem), followed by COP purification.

The fluorescent oligonucleotides, respectively, synthesized and purified by means of said method were quantified by a spectrometer and dried with a vacuum condenser. Thereafter, they were prepared by DW into the stocks of 1 nmol/ul concentration, which were then kept in a freezer. When in use, it was diluted with hybridization solution to appropriate concentration.

The types, base compositions and characteristics of fluorescent oligonucleotides, respectively, were as follows:

Flu. 1: 5′-TAC GGG TCA ATG TCC ATG CCC CAA-fluorescein-3′ (24 mer)

Consists of the complementary base sequence of DMT 1 and No. 1.

Flu. 2: 5′-TAC GGG TCA ATA ACC ATG CCC CAA-fluorescein-3′ (24 mer)

Consists of the complementary base sequence of DMT 2 and No. 2

Flu. 3: 5′-fluorescein-GTG GTT TCA CCT ACG ACG ACA CTA-3′ (21 mer)

Flu. 4: 5′-fluorescein-GAA TAG AAG GAA AGA AGT CAG-3′ (21 mer)

Consists of the complementary base sequence of Pyr. 1 and Chol. 1.

Flu. 5: 5′-fluorescein-CCA GCC ACA GGC CCA GG-3′ (17 mer)

Consists of the complementary base sequence of Chol. 2 and No. 3.

Flu. 6: 5′-fluorescein-CTC ACC AGC ATA TCC CCA TAA-3′ (21 mer)

Consists of the complementary base sequence of Chol. 3 and No. 4.

Flu. 7: 5′-fluorescein-TCC ATC AAC TGT GAG GCG GAA-3′ (21 mer)

Consists of the complementary base sequence of Chol. 4 and No. 5.

### EXAMPLE 2

### Preparation of Oligonucleotide Array

A glass slide in general use was treated with 2N HCl for five minutes, followed by washing it with distilled water for three times. Then, the moisture was removed by treating it with acetone for one minute. For the purpose of firmly bonding the polymers (i.e., the attachment layer), the surface thereof was treated with bonding silane (dimethyldicholorosilane), after which was kept at room temperature for future use.

Onto the slide surface treated with bonding silane, the UV solidified resin (the resin solidifies only if it is irradiated with UV (commercially sold)) was evenly applied at thickness of 0.2-0.3 mm. In this manner, the attachment layer in the state of fluidity was formed, onto which were spotted at 0.2˜1 ul, respectively, with DMT of various concentrations prepared according to Example 1, oligonucleotides modified with pyrene or cholesterol, and generic unmodified oligonucleotides.

Thereafter, while maintaining the slide in the direction in which the surface spotted with oligonucleotides was facing downward, it was left out in air for 3-5 minutes. Then, the UV solidified resin was solidified by irradiating with UV at 312 nm for 30 seconds to 1 minute. In this manner, the oligonucleotides were thus attached thereto. In some cases, for complete polymerization (solidification) of said UV solidified resin, the aqueous solution layer was formed by adding distilled water at 0.5 ul at the location where oligonucleotides were spotted. Then, they were irradiated for one minute with a UV lamp for secondary solidification.

The oligonucleotide array prepared by the above method was dried for two hours in air. Then, for removing non-bonded oligonucleotides therefrom, it was washed with 1×TE buffer solution of pH 8 for three times, or dipping the same into 0.2% SDS solution and washing it with distilled water for two minutes for two times. It was then dried in air for use in hybridization.

### EXAMPLE 3

### Hybridization

For DMT oligonucleotide, hybridization was carried out as follows: To 5×SSC, 0.5% SDS hybridization solution, complementary and noncomplementary fluorescent oligonucleotides were diluted in appropriate amounts. The solution was spotted at 0.5 ul respectively to the locations where the oligonucleotides had been spotted. To prevent evaporation of the solution, the slide was placed in a hybridization cassette in which humidity was maintained with 2×SSC or 1×TE buffer solution at 30° C. for three hours. For washing the slide, it was treated twice with 2×SSC, 0.1% SDS solution for 10 minutes, respectively. Then, it was washed five times with 1×TE buffer solution at pH 8. The fluorescence was measure and analyzed by using Storm® of Molecular Dynamics, Inc. (USA). In the present invention, it was analyzed with scan resolution of 200 um per pixel. When spotted with 0.5 ul, it resulted in surface area of approximately 3 mm², which corresponded to 75 pixels/spot (pixel/spot). The fluorescent sizes of respective pixels were quantified to the precision of 16-bits. The data were analyzed with ImageQuaNT v4.0 analysis program.

Moreover, the hybridization tests with respect to oligonucleotides modified by pyrene and/or cholesterol were carried out as follows: First, to the 5×SSC, 0.2% SDS hybridization solution, the respective fluorescent oligonucleotides were diluted to 10 pmol/ul. 10 ul of the solution (100 pmol) was spotted to the slide bonded with oligonucleotides according to the method of Example 2. The cover glass (18×18 mm) was placed over the slide without forming bubbles. The slide was placed into the hybridization cassette, and while maintaining humidity, the hybridization was carried out by treatment therein for two hours at 50° C. With respect to the slide after completing the hybridization, the cover glass was removed by using wash solution I (2×SSC, 0.2% SDS). It was treated by dipping the same into the new batch of wash solution I for 30 minutes at 37° C. Then, it was washed by a step-by-step basis by dipping the slide for three minutes at room temperature, respectively, in wash solution (II) (0.2×SSC, 0.2% SDS), wash solution m (0.2×SSC), and distilled water. Thereafter, it was completely dried in air, and the fluorescence thereof were measured and comparatively analyzed by ScanArray 5000 of GSI Lumonics, Inc. There, the resolution was 10 um, laser power was 50˜55%, and PMT grain was 60˜65%. The results of hybridization are shown in Tables 7, 8 and 9.

### EXAMPLE 4

### Confirmation of Attachment of Oligonucleotide

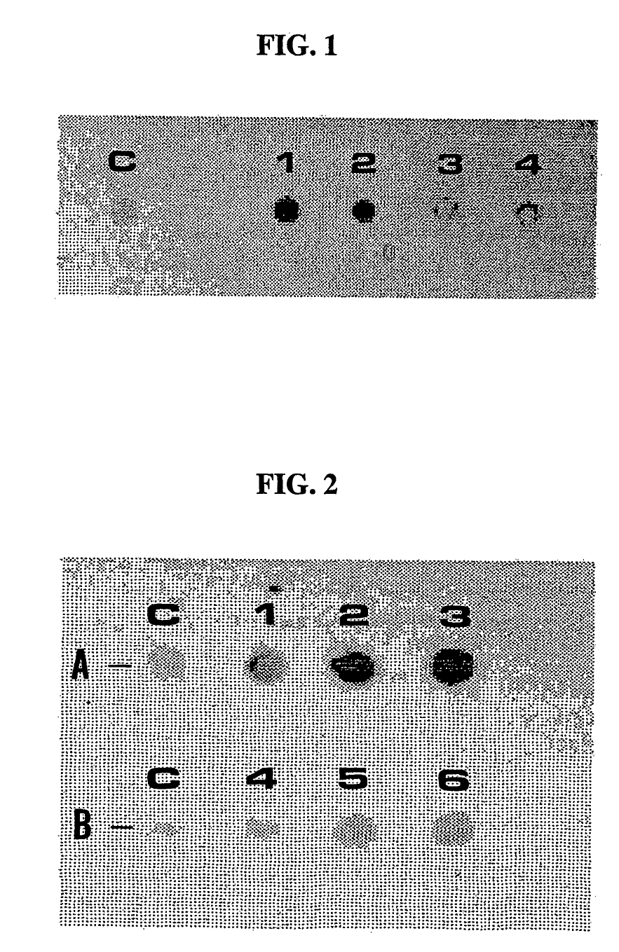

In order to confirm the attachment of oligonucleotide on the support by way of hydrophobic DMT, two types of DMT oligonucleotides, DMT 1 and DMT 2 (Spots 1 and 2 of FIG. 1), and generic oligonucleotides Nos. 1 and 2 of the same base sequence as the DMT oligonucleotides (Spots 3 and 4 of FIG. 1) as attachment layers were respectively spotted onto the surface of the support applied with the polymers at 30 pmol/spot (0.3 ul). Then, the hybridization was carried out by means of the respective complementary fluorescent oligonucleotides (Flu. 1 and Flu. at 2) at 25 pmol. The results thereof are shown in FIG. 1. As a control group, “C” is the result obtained from spotting with 1×TE buffer solution at 0.3 ul without hybridization. The results of FIG. 1 show that DMT oligonucleotide was well attached onto the support by DMT and that hybridization was possible therein.

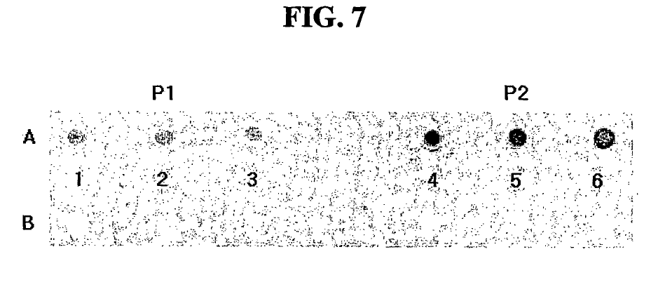

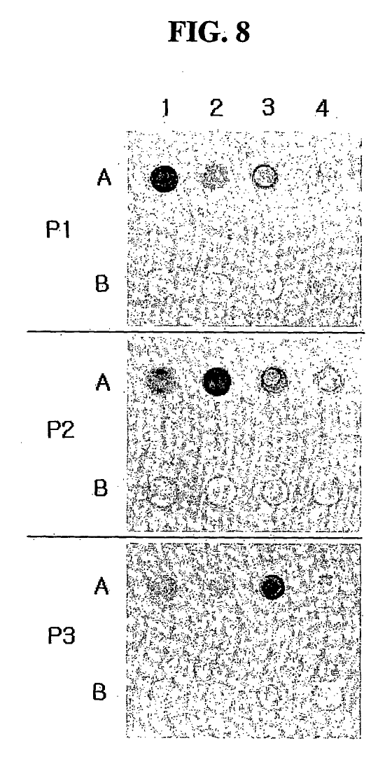

With respect to the oligonucleotide in which its 5′ terminal had been bonded with pyrene or cholesterol, the tests were carried out by said methods. The results thereof are shown in FIGS. 7, 8 and 9.

FIG. 7 is a photograph, which shows the results of hybridization by complementary and non-complementary fluorescent oligonucleotides after attaching pyrene oligonucleotide and generic oligonucleotide onto the supports, respectively.

Pyrene oligonucleotide (Pyr. 1) was spotted to 1˜6 of lane A, and the generic oligonucleotide (No. 4) to 1, 2, 4 and 5 of lane B. Further, to spots 3 and 6 of lane B, which were used as controls, the 1×TE buffer solution without oligonucleotide was spotted at 0.2 ul.

Moreover, in P1, the hybridization was carried out with a fluorescent oligonucleotide, which was non-complementary to pyrene oligonucleotide (Pyr. 1) but complementary to the generic oligonucleotide (No. 4). In P2, the hybridization was carried out with the fluorescent oligonucleotide (Flu. 4), which was complementary to Pry. 1.

Moreover, in P1, hybridization was carried out with a fluorescent oligonucleotide, and in P2 and P3, with Flu. 5 and Flu 6, respectively.

As a result, cholesterol oligonucleotide (Chol. 1) reacted specifically with the fluorescent oligonucleotide (Flu. 4), the sequence of which was complementary thereto, and the cholesterol oligonucleotides (Chol. 2 and Chol. 3), respectively, reacted specifically with the fluorescent oligonucleotides (flu. 5 and Flu. 6), the sequences of which were complementary thereto, respectively.

Through comparison of A with B of the respective lanes of P1P3, irrespective of the base composition and length of each oligonucleotide, the oligonucleotides were shown to become attached by way of cholesterol in such a manner to allow specific hybridization on the support.

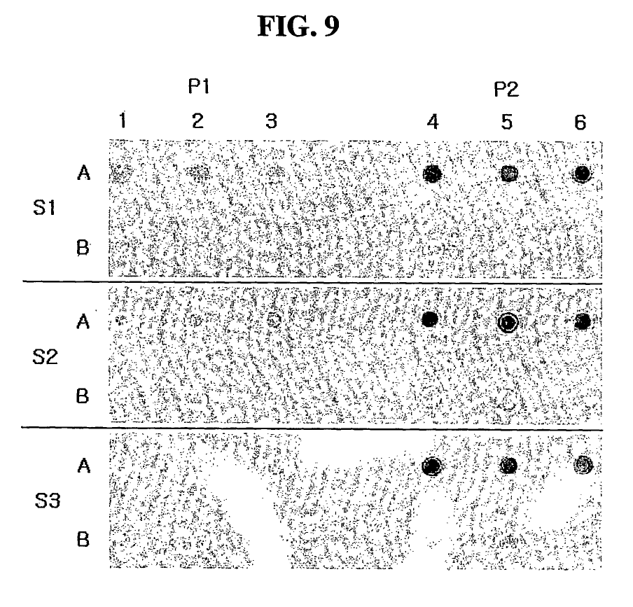

FIG. 9 is a photograph, which shows the results of hybridization by complementary and non-complementary fluorescent oligonucleotides after attaching three types of cholesterol oligonucleotides and generic oligonucleotides onto the supports.

Cholesterol-oligonucleotide (Chol. 2) was attached to S 1—lane A; generic oligonucleotide (No. 3) to S1—lane B; cholesterol-oligonucleotide (Chol. 3) to S2—lane A; cholesterol-oligonucleotide (Chol. 3) was attached to S2—lane A; generic oligonucleotide (No. 4) to S2-lane B; cholesterol-oligonucleotide (Chol. 4) to S3—lane A; and generic oligonucleotide (No. 5) to S3—lane B.

In spots B3 and B6 of S1, S2, and S3, as controls, 1×TE without oligonucleotide was spotted at 0.2 ul, respectively.

Moreover, to each spot, the hybridization was carried out with the fluorescent oligonucleotide as follows:

S1-P1: Flu. 4 Fluorescent oligonucleotide hybridization;

S1-P2: Flu. 5 Fluorescent oligonucleotide hybridization;

S2-P1: Flu. 7 Fluorescent oligonucleotide hybridization;

S2-P2: Flu. 6 Fluorescent oligonucleotide hybridization;

S3-P1: Flu. 4 Fluorescent oligonucleotide hybridization; and

S3-P2: Flu. 7 Fluorescent oligonucleotide hybridization;

In contrast to generic oligonucleotide (Lane B), cholesterol oligonucleotide (Lane A) was attached onto the support and allowed specific hybridization therein (comparison of 1, 2, and 3 of Lane A with 4, 5, 6 thereof). Through comparison with each “S”, it was reconfirmed that the attachment occurred irrespective of the types (base composition and length) of cholesterol-oligonucleotides. Moreover, the reproducibility therein could be confirmed by way of 4, 5, and 6 of lane A.

### EXAMPLE 5

### Comparison of Hybridization of Complementary and Non-Comlementary Fluorescent Oligonucleotides

To confirm the specific hybridization of oligonucleotides attached to the support, the hybridization was carried out by differentiating the concentrations of the complementary and non-complementary fluorescent oligonucleotides after the attachment of DMT oligonucleotide.

In FIG. 2, the oligonucleotide (DMT 1) was respectively spotted at Sopmol/spot (0.5 ul). Then, the hybridization was carried out in lane A with the complementary fluorescent oligonucleotide (Flu. 1), respectively at 10, 50, 100 pmol (spot 1, 2 and 3). Moreover, the hybridization was also carried out in lane B with the non-complementary fluorescent oligonucleotide (Flu. 3), respectively at 10, 50, 100 pmol (spot 4, 5 and 6). As a control, “C” was left in the state without hybridization. As shown in the results of FIG. 2, the hybridization occurred when the complementary fluorescent oligonucleotide was added. Moreover, the higher the concentration of the complementary fluorescent oligonucleotide, the better was the hybridization therein.

### EXAMPLE 6

### Confirmation of Reproducibility

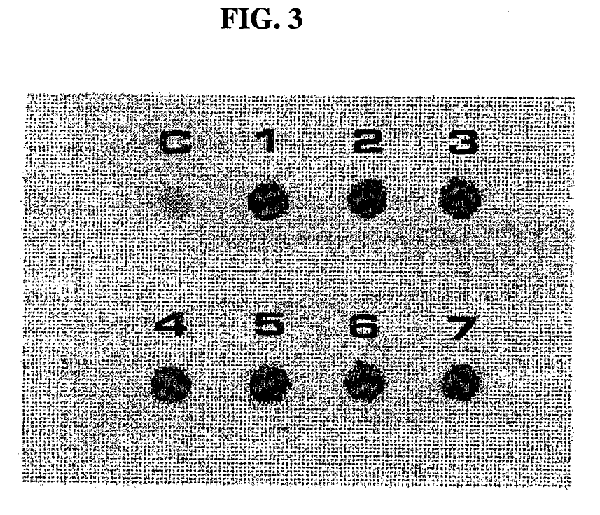

After spotting the oligonucleotide (DMT 1) at 0.5 ul (50 pmol), eight of them, respectively, the hybridization was carried out with 50 pmol of the complementary fluorescent oligonucleotide (Flu. 1). The results of the seven samples (spots 1-7 of FIG. 3) were compared for confirming reproducibility. “C” was a control group, in which hybridization had been carried out after spotting with DMT oligonucleotide. The results thereof are shown in FIG. 3.

With respect to pyrene oligonucleotide and cholesterol oligonucleotide, the tests for conforming reproducibility were carried out with three spots of equivalent concentrations as in FIGS. 7, 8 and 9. From the results thereof, the reproducibility therein could be confirmed.

### EXAMPLE 7

### Confirmation of Attachment Stability

For confirming stability of the state of attachment by DMT, the slides after the confirmation of attachment were treated for 18 hours at room temperature while dipping them into the 1×TE buffer solution. Thereafter, the fluorescent sizes were once again measured.

Electrophoresis was carried out by using the slides which had been used for confirmation of reproducibility. There were no differences in fluorescent size. In other words, it was shown that there was indeed stability in bondings.

### EXAMPLE 8

### Comparison of Attachment and Hybridization Efficiency

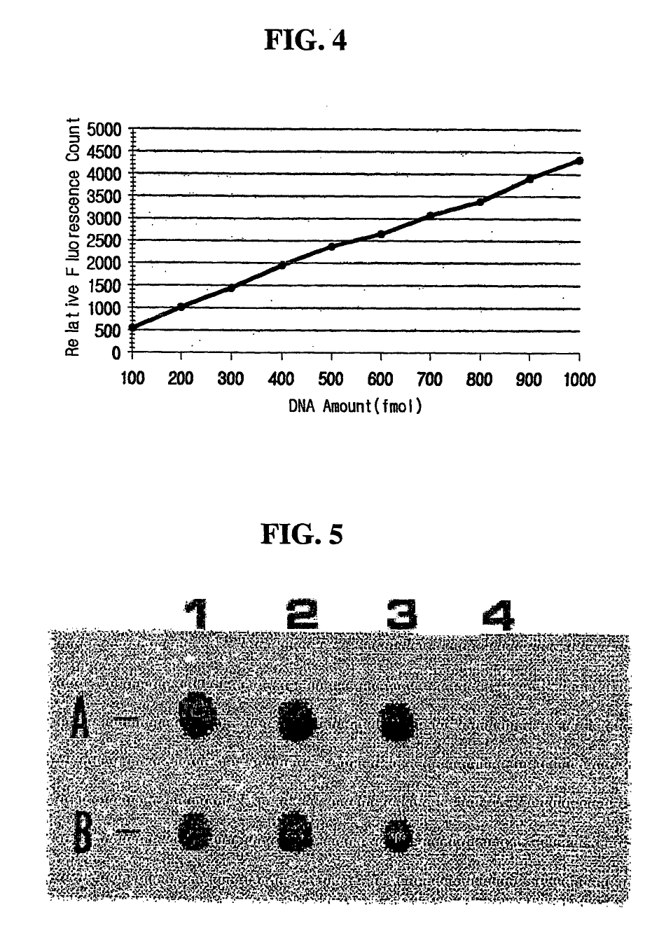

After spotting with oligonucleotide (DM 1) at 100 pmol (lane A) and 50 pmol (lane B), four of each, the hybridization was carried out with a complementary fluorescent oligonucleotide (Flu. 1) at 100, 50, 25, and 0 pmol, (1, 2, 3, and 4) respectively. The results thereof were compared, which are shown in FIG. 5. The respective fluorescent values were compared with the standard curve of FIG. 4. The results of attachment and hybridization density as calculated are shown in Table 1.

TABLE 1

| | | Hybridization amount | | |
|---|---|---|---|---|
| | | 100 pmol | 50 pmol | 25 pmol |
| Amount of DMT oligonucleo- | 100 pmol | 420 | 400 | 260 |
| tide used in attachment | 50 pmol | 430 | 290 | 200 |
| (Unit: fmol/mm²) | | | | |

According to the present invention, with respect to the oligonucleotide as attached, the hybridization therein was possible up to 430 fmol/mm².

With respect to the method of attaching oligonucleotide onto a support, according to the present invention, it is not necessary to have pre-treatment of attaching a separate linker onto the support and to oligonucleotide.

Moreover, with respect to the method of attaching oligonucleotide according to the present invention, due to its high bonding efficiency, the method can simultaneously attach various types of oligonucleotides.

Moreover, the method of attaching oligonucleotide according to the present invention provides significantly higher hybridization efficiency as compared to that of the prior art, and thus it is useful in various areas, such as diagnosis.

With respect to the method attaching oligonucleotide according to the present invention, and the oligonucleotide array prepared by said method, they allow specification of the direction of an oligonucleotide array in the direction of 5′→3′ from the support, or in the direction of 3′→5′ from the support. As such, they provide high hybridization efficiency and are more useful as compared to the conventional methods.

Moreover, with respect to the method attaching oligonucleotide according to the present invention, and the oligonucleotide array prepared by said method, they involve less steric hindrance as compared to non-specific bonds between the conventional support and an oligonucleotide.

What is claimed is:

1. A method of attaching oligonucleotide onto a support, which comprises the steps of applying a hydrophobic attachment layer, which can be solidified under certain conditions, onto the support; spotting a solution of oligonucleotide with its 3′- or 5′-terminal bonded to a hydrophobic group onto a certain portion of said attachment layer; and solidifying said attachment layer.

2. A method of attaching oligonucleotide onto a support, which comprises the steps of fluidifying a solidified hydrophobic attachment layer as applied onto the support under certain conditions; spotting a solution of oligonucleotide with its 3′- or 5′terminal bonded to a hydrophobic group onto a certain portion of said attachment layer; and solidifying said attachment layer.

3. The method of attaching oligonucleotide onto a support, according to claim 1 or 2, wherein said oligonucleotide bonded to a hydrophobic group is a PCR amplified product from a primer with its 5′-terminal bonded to a hydrophobic group.

4. The method of attaching oligonucleotide onto a support, according to claim 1 or 2, wherein said attachment layer is of polymers formed by polymerization or condensation polymerization.

5. The method of attaching oligonucleotide onto a support, according to claim 1 or 2, wherein said hydrophobic group, in one or more, is selected from the group consisting of a dimethoxytrityl group, a pyrene group, and cholesterol.

6. The method of attaching oligonucleotide onto a support, according to claim 3, wherein said attachment layer is of polymers formed by polymerization or condensation polymerization.

7. The method of attaching oligonucleotide onto a support, according to claim 3, wherein said hydrophobic group, in one or more, is selected from the group consisting of a dimethoxytrityl group, a pyrene group, and cholesterol.

8. An oligonucleotide array made by a method according to one of claims 1-7.
